(19) United States
(12) Patent Application Publication
(10) Pub. No.: US 2020/0093929 A1
(43) Pub. Date: Mar. 26, 2020

(54) PHARMACEUTICAL COMPOSITION FOR THE PREVENTION AND TREATMENT OF MEMORY AND COGNITIVE DEFICITS RELATED TO AGEING

(71) Applicant: IGEA PHARMA N.V., Hoofddorp (NL)

(72) Inventors: Gianfranco LIGURI, Firenze (IT); Ilaria SERNESI, Bagno a Ripoli (FI) (IT); Massimo STEFANI, Campi Bisenzio (FI) (IT)

(73) Assignee: IGEA PHARMA N.V., Hoofddorp (NL)

(21) Appl. No.: 16/484,034

(22) PCT Filed: Feb. 8, 2018
(86) PCT No.: PCT/IB2018/050794

§ 371 Date: Aug. 6, 2019

(30) Foreign Application Priority Data
Feb. 8, 2017 (IT) 102017000013599

(51) Int. Cl. A61K 47/54; A61K 38/06; A61K 31/7048; A61K 45/06; A61K 9/00; A61P 25/28; A61K 31/59; A61K 31/714; A61K 31/355; A23L 33/10

(52) U.S. Cl. CPC A61K 47/542; A61K 38/063; A61K 31/7048; A61K 45/06; A61K 9/0053; A23V 2002/00; A61K 31/59; A61K 31/714; A61K 31/355; A23L 33/10; A61P 25/28

(57) ABSTRACT

A pharmaceutical composition for the prevention and treatment of human cognitive deficit includes oleuropein or its derivatives and glutathione conjugated with a carboxylic acid. The carboxylic acid component of the glutathione-carboxylic acid conjugate is any saturated or unsaturated aliphatic carboxylic acid, containing from 2 to 22 carbon atoms.

### TECHNICAL FIELD

The present invention relates to the field of substances for medical use; in particular, it relates to the use of oleuropein and bioavailable glutathione derivatives in the therapeutic and/or prophylactic treatment of cognitive deficits and the decline in neurological performance related to Alzheimer's Disease (AD), Parkinson's disease and other neurodegenerative diseases in humans.

More specifically, the present invention relates to a formulation containing thioesters of glutathione with carboxylic acids, in association with polyphenols of Olea europea. The formulation and method described by the present invention can be used in order to prevent, halt the progression or treat cognitive and mnestic impairment in human subjects at risk of developing Alzheimer's disease or other neurodegenerative processes. Bioavailable glutathione, able to counteract oxidative stress and apoptosis in the early stages of neurodegenerative pathogenesis, and oleuropein, o polyphenol active against the aggregation of the neurotoxic amyloid peptide AP (1-42) and capable of activating the autophagic process, act synergistically counteracting neuronal loss and preserving the integrity and correct functioning of the central nervous system.

### BACKGROUND ART

It is known that a number of age-related degenerative diseases in humans are characterised by cognitive impairment due to the progressive loss of function of specific structures in the central nervous system.

Alzheimer's disease is the most common age-related neurodegenerative disease in humans, leading to progressive severe disability and ultimately to death. With the ageing of the world population, it is estimated that the number of people affected by AD will grow from about 46,000,000 cases estimated today to about 131,500,000 estimated by 2050 (World Alzheimer Report 2015, Alzheimer's Disease International, London, Publisher). From the neuropathological point of view, the disease is characterised by the presence in the brain parenchyma of extracellular amyloid deposits (plaques), consisting mainly of amyloid beta peptide (1-42) and its derivatives and of neurofibrillary tangles of tau hyperphosphorylated protein in neurons of encephalic regions affected by the degenerative process. The concentration of glutathione (GSH), the most abundant intracellular non-protein thiol and important antioxidant, decreases with age and in some age-related diseases. Numerous studies show that a reduction in the GSH content is involved in AD disease in humans (Ann NY Acad. Sci. 2004 Jun; 1019: 346-9; J. Nutr. March 1, 2004 vol. 134 No. 3 489-492; Journal of Blood Medicine 2013: 4 31-38). Early stages during the pathogenesis of AD and other neurodegenerative diseases, such as Huntington's chorea and Parkinson's disease, are in fact characterised, among other things, by a strong oxidative stress in which an excess of free radicals causes a significant depletion of intracellular glutathione that is accompanied by deterioration of the affected neurons. It is believed that specific intracellular signalling pathways, in which caspase proteins participate, activated by the disruption in the oxidative-reductive homeostasis, trigger the neuronal apoptotic process that eventually leads to neuronal atrophy, loss of encephalic mass and deterioration of neuro-psychomotor performance.

Glutathione, which is the main antioxidant defence in the body, is one of the metabolites with the highest concentration in mammalian tissue cells. Its concentration is between 1 and 10 mM depending on the cell type. When taken orally, glutathione as such is unable to cross the alimentary canal intact as it undergoes a hydrolytic cleavage catalysed by digestive enzymes, in particular by pancreatic peptidase and the intestinal wall (Witschi A, et al., Eur J Clin Pharmacol. 1992;43(6):667-9.). Furthermore, its bioavailability to cells is reduced even if administered parenterally as it is unable to cross the cell membrane due to the absence of specific transporters and its hydrophilicity. In recent years, glutathione forms for which greater bioavailability has been demonstrated for tissues, including nervous tissue have been described and are available on the market. In particular, glutathione derivatives conjugated with aliphatic carboxylic acids at the level of the cysteine residue have proved to be particularly bioavailable, and therefore capable of significantly increasing the intracellular concentration of the tripeptide even when they are supplied at concentration levels of orders of magnitude lower than that reached within the cells (A. Pensalfini et al./Free Rad Biol Med 2008, 44 1624-1636; Okun J G, et al., J Inherit Metab Dis. 2004;27(6):783-6.). This effect is believed to be the result of a trapping process by the cells, which do not allow the release of glutathione penetrated as thioesters and subsequently hydrolysed by intracellular thioesterases. Conjugates of glutathione with commercially available aliphatic carboxylic acids, which have been shown to be highly bioavailable from preclinical and clinical studies, are S-acetyl glutathione, SAG) and glutathione derivatives conjugated with long-chain fatty acids (Long-chain fatty acid-glutathione, LCFA-SG). The latter, in particular glutathione derivatives with omega-3 fatty acids, such as S-linolenoyl glutathione, have proved particularly effective in counteracting oxidative stress in the neurons of patients with AD (M. Zampagni et al., Free Radical Biology & Medicine 2012, 52: 8, 1362-71). In particular, glutathione conjugates with polyunsaturated fatty acids have proved particularly effective in preventing cell death in cultures of human neurons exposed to oxidative stress induced by hydrogen peroxide, Abeta peptide (1-42) and ultraviolet radiation (Wright D et al., Photochemistry and Photobiology, 2013, 89: 442-452; Pensalfini A et al. Free Rad Biol Med 2008, 44 1624-1636). Hence the bioavailable glutathione forms, according to the present invention, are the thioester derivatives of glutathione with aliphatic carboxylic acids containing from 2 to 22 carbon atoms. The same compounds have proven themselves able to increase the average lifespan in Caenorhabditis elegans (Cascella R et al. Free Radical Biology and Medicine 73(2014),127-135).

Oleuropein (OLE) is a secoiridoid polyphenol of plant origin found in plants of the Oleaceae family and in particular in Olea Europaea, the common olive tree in its many varieties in which it can be present both as a glucoside and in the form of an aglycone. Oleuropein, in its glycated form, is a water-soluble chemical species with a molecular weight of 540.51, brute formula C₂₅H₃₂OR₁₃ and molecular formula:

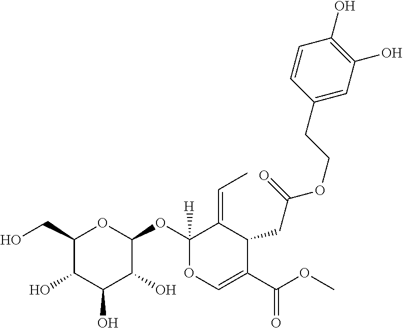 [SMILES: C/C=C1/[C@H](O[C@@H]2O[C@H](CO)[C@@H](O)[C@H](O)[C@H]2O)OC=C(C(=O)OC)[C@H]1CC(=O)OCCc1ccc(O)c(O)c1]

Following hydrolysis by glycosidases present in olives during ripening and pressing for the production of extra virgin olive oil, the glycosidic component of oleuropein is widely released, so that oleorupein aglycone, which is more hydrophobic than the glycosylated form, is predominantly present in the oil. A similar process occurs in the alimentary canal by the intestinal glycosidases.

Oleuropein aglycone, with a molecular weight of 376.41, brute formula C₂₀H₂₄OR₇ and molecular formula

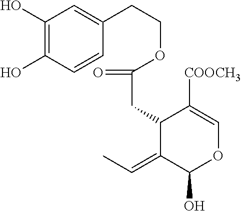

can be prepared from glycated oleuropein through in vitro deglycosylation by β-glycosidase. Rigacci S et. al. have shown that oleuropein aglycone interferes with the aggregation of human amylin (hIAPP), and thus significantly reduces the high toxicity of hIAPP to a human pancreatic cell line. Following this and other experimental and clinical evidence, oleuropein aglycone has been proposed for the treatment of type 2 diabetes mellitus. (Rigacci S et al., J. Nutr. Biochem. 21, 726-735, 2010). Recently, especially thanks to other research conducted at the University of Florence, it has been demonstrated experimentally that oleuropein modifies the aggregation of the beta-amyloid peptide ABeta1-42 (Aβ (1-42),) hindering the formation of toxic oligomeric species.

Furthermore, oleuropein, added to preformed fibrillar Aβ (1-42) aggregates, inhibits their release of toxic oligomers and neutralises the residual toxicity associated with these molecular species. In particular, Aβ (1-42) aggregates in non-toxic amyloid assemblies in the presence of the natural polyphenol oleuropein aglycon (Rigacci S et al., Curr. Alz. Res. 8:841-52, 2011).

Other studies conducted both in vivo and in vitro revealed the great potential of the phenolic component present in Olea europaeatissues, mainly oleuropein and oleocanthal in non-glycated forms, in counteracting the amyloid aggregation and neurotoxicity of the prefibrillar AP peptide aggregates (Rigacci S et al., Expert Rev Neurother, 15(1):41-52, 2015). These studies have also provided evidence on the pathogenetic mechanisms involved in the onset and progression of Alzheimer's disease on which these polyphenols act (amyloid precursor protein processing, extracellular plaque deposition produced by Aβ peptide aggregation and intracellular neurofibrillary aggregates of tau protein, autophagy dysfunction, neuro-inflammation) (Rigacci S et al. Oncotarget 2016, 6(34):35344-57; Casamenti F et al., J Alz Dis 2015 45:679-88; Casamenti F et al., Exp Rev Neurother 2016 Oct 20:1-14; Rigacci, S. Adv Exp Med Biol 2015, 863:1-20). The in-vivo results were obtained in mouse models of cerebral amyloid deposition (Grossi C et al., PLoS One 2013, 8: e71702; Luccarini I et al. Neurobiol. Ag. 2015, 36: 648-63) or in rats treated with intracerebral injection of preformed aggregates in the presence or absence of oleuropein aglycone (Luccarini I et al. Neurosci. lett. 2014, 558: 67-72,). A large body of data suggests that amyloid aggregation, and in particular the first phase of peptide oligomerisation, combined with dysfunction of the autophagic process (Menzies FM et al. Nature Rev Neurosci 2015, 16: 345-57) represents the main cause of the disease.

It is therefore evident that the research to find any drugs that are active against AD should focus on the identification of molecules that are capable of restoring correct proteostasis by inhibiting the formation of toxic species at the onset of amyloid aggregation and the restoration of oxidation-reductive homeostasis, in particular of physiological levels of intracellular glutathione.

The scientific literature has in recent years described numerous potential remedies to prevent and treat neurodegenerative conditions. These include synthetic drugs, biotherapeutic products (monoclonal antibodies) and nutraceuticals extracted from natural sources, as well as a significant number of new candidate molecules, which are currently undergoing clinical validation.

To date, however, no effective pharmacological treatments for preventing or treating Alzheimer's disease and other human neurodegenerative diseases that are validated and included in official guidelines have been described.

Therefore, the object of the present invention is to formulate the combination of an active principle capable of stimulating the autophagic process and of inhibiting the formation of neurotoxic amyloid aggregates and bioavailable derivatives of glutathione capable of both combating the oxidative stress that characterises the early stages of the pathogenesis of diseases such as Alzheimer's and Parkinson's disease and restoring the physiological levels of this tripeptide in brain tissues.

Recently numerous studies have highlighted the correlation between specific nutritional deficiencies, in particular of Vitamins D_{3,} E, B₆ and B_{12,} and increased risk of the manifestation of cognitive impairment and of the incidence of neurodegenerative diseases (Littlejohns T J, et al., Neurology. 2014 Sep 2;83(10):920-8; Morris M C, et al., JAMA. 2002; 287(24):3230-3237). It has also been reported that some plant extracts, such as extracts from Bacopa monnieri, of Curcuma longa, etc., have a neuroprotective action (Liu X, et al., Pharmacology Biochemistry and Behavior 2013: 110, 224-230; Mishra S, et al., Ann Indian Acad Neurol. 2008 Jan-Mar; 11(1): 13-19.). Therefore it may be useful to associate one or more of these active principles for the treatment of Alzheimer's and Parkinson's disease, Huntington's chorea and other neurodegenerative processes to the formulation to which the present invention refers.

### Disclosure of Invention

The inventors have surprisingly found that the association between oleuropein and bioavailable forms of glutathione is clearly more effective than the components taken separately in counteracting and normalising the biochemical alterations and the decline in memory and in cognitive performance that occur in the early stages and in the progression of Alzheimer's disease and other neurodegenerative diseases in experimental animal and cell models. Based on current knowledge and experimental evidence, this effect is believed to be due to the synergy that is produced by the combined action of oleuropein (or its metabolites such as hydroxytyrosol), an effective autophagy stimulator and an inhibitor of the Aβ (1-42) peptide aggregation in neurotoxic prefibrillar complexes, and glutathione in a bioavailable form, which is effective in counteracting the oxidative stress associated with the neurodegenerative process. The extraordinary effectiveness of the two active principles in counteracting cognitive deficits and neurodegeneration therefore appears to stem from the reciprocal enhancement resulting from their synergistic cooperation in counteracting the cellular biochemical mechanisms underlying the initial phases of the neurodegenerative process.

Therefore, the aim of the present invention is the use of oleuropein and bioavailable glutathione derivatives in the prevention and/or therapeutic treatment of the cognitive deficits typical of Alzheimer's disease and of other neurological diseases related to ageing.

Furthermore, pharmaceutical compositions for the treatment of Alzheimer's disease and other ageing-related neuro-pathologies are also the objective of this invention. Said compositions would comprise the association in a pharmaceutically acceptable form of oleuropein, glutathione conjugated with carboxylic acids and possibly one or more ingredients that are active in counteracting neurodegeneration associated with ageing, or endowed with nootropic or neuroprotective activity, or essential nutrients whose deficiency is correlated with the risk of neurodegeneration. Compounds of this type are, by way of an example, the following: curcumin, bacosides from Bacopa monnieri, vitamin D_{3,} vitamin B_{6,} astaxanthin, lutein, tocopherols, tocotrienols, vitamin B_{12,} choline, betaine.

This invention provides a solution, in the form of a pharmaceutical, nutraceutical or medical device, for counteracting hair loss or promoting its regrowth. The solution provided by this invention is based on the synergy between the free radical inactivation activity exerted by the thioesters of glutathione, and the anti-inflammatory activity exerted by the secoiridoids of European olea.

According to this invention, the composition that is the subject of the invention can be used to prepare a pharmaceutical formulation, a pharmaceutical remedy, a cosmetic product, a medical device, a nutraceutical preparation or a functional food for preventing or treating hair loss.

The combination, in the same preparation, of a component that is highly effective at combatting oxidative stress and a component capable of fighting the inflammatory process—both alterations that are characteristically found in in the hair bulbs of skin in alopecia—has shown itself to be surprisingly superior to a simple additive effect of both components.

Being two components of bioavailable derivatives of glutathione and oleuropein respectively, the unexpected synergy of their combination in counteracting hair loss and in stimulating their regrowth, can be ascribed on the one hand, to their shared antioxidant characteristics, and on the other to the proven ability of glutathione to restore redox homeostasis, which is typically altered in the inflammatory process. In view of the effects observed, and on the basis of recent publications about the mechanisms of action involved in their activity, an interaction between these two substances can be suggested in the same biochemical, metabolic and signalling pathways—intracellularly—with the overall effect of strengthening their biological effects (British Journal of Nutrition (2006), 96, 811-819 DOI: 10.1017; Proc. Natl.Acad.Sci.USA 103:13086-13091; 2006.). The bioavailable derivative of glutathione can be obtained from a direct reaction between glutathione and S-acyl-Coenzyme A or via a conjugation reaction between an activated carboxylic acid and glutathione in its reduced form. This latter synthetic route is especially indicated for manufacturing glutathione derivatives suitable for industrial use, and is described in WO2013/068964A1.

Oleuropein can also be obtained by synthesis (e.g., WO96/14064), although the most convenient production method for industrial use is extraction from its natural sources. Particularly useful for this purpose are the leaves of European olea (e.g. US2003-0017217) or plant waters produced in the olive oil production process, due to their richness in this secoiridoid polyphenol. The powder obtained by evaporating the solvent from the extract with a hydro-alcoholic solution of these materials and further purification steps can provide variable percentages, of between 5% and 90% of oleuropein, in both glycated and aglycone forms, along with variable proportions of other polyphenols and terpenoids.

The sources reported herein for the production of thioesters of glutathione and oleuropein are reported for the sole purpose of description, and do not exclude any other source or methods for obtaining the two components, for the purposes of this invention. According to this invention, the composition for preventing or treating hair loss can be made in the form of a pharmaceutical formulation, a nutraceutical preparation, a medical device, a cosmetic product. When the composition is prepared as a pharmaceutical remedy, the preferred routes of administration are the oral route or the parenteral route and the composition may comprise a carrier, a lubricant, a humectant, a sweetener, a flavouring agent, an emulsifier and one or more pharmaceutically acceptable preservatives, chosen from those commonly used for these purposes and known to those who are skilled in the art.

When the composition that forms the subject of this invention is prepared as a cosmetic formulation, the preferred method of administration is topical application in the form of liquid lotion, ointment, solution, suspension, emulsion, paste, ointment, gel, or cream, powder, soap or aerosol, and the site of application is the external surface of the scalp or a part thereof. The cosmetic formulation may contain, in addition to the two components responsible for the anti-alopecia activity described herein, substances suitable for promoting their penetration into the lower layers of the skin, such as alcohols or glycols or other organic compounds with a reduced polarity, and other additives such as antioxidants, stabilizers, vasodilators, vitamins, pigments, flavourings and carriers. Said additives can be selected from a multitude of compounds from those familiar to experts in the art of formulating cosmetic products.

When the composition is prepared as a medical device, the preferred mode of administration is the multi-intracutaneous injection (Micro-needling or similar techniques), ionophoresis or topical application onto the external surface of the scalp or part thereof, in the form of liquid lotion, ointment, solution, suspension, emulsion, paste, ointment, gel , of cream, powder, soap or aerosol.

When the composition is prepared as a nutraceutical preparation, it may include a carrier, a sweetener, one or more antioxidants, one or more mineral salts, one or more vitamins, one flavouring agent, one emulsifier and one or more preservatives, chosen from amongst those commonly used as food additives and known to those skilled in the art.

In order to prevent hair loss and promote hair growth in the scalp, the simultaneous administration of the two active principles contained in the composition forming the subject of this invention, by both topical and systemic administration, via concomitant administration, has been particularly effective, for a period of not less than one month, for a scalp lotion and a nutritional supplement.

### DETAILED DESCRIPTION OF THE INVENTION

A pharmaceutical composition according to the invention for the prevention and treatment of human cognitive impairment comprises oleuropein or its derivatives and glutathione conjugated with a carboxylic acid, of molecular formula

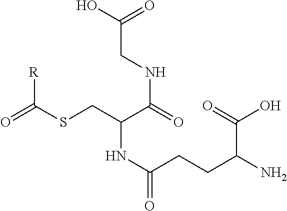

wherein R=aliphatic carboxylic acid residue and the carboxylic acid component of the glutathione conjugate—carboxylic acid is any saturated or unsaturated aliphatic carboxylic acid containing from 2 to 22 carbon atoms.

The carboxylic acid component of the glutathione-carboxylic acid conjugate usefully consists of ethanoic acid or a omega-3, omega-6 or omega-9 unsaturated aliphatic carboxylic acid containing 18 carbon atoms.

Oleuropein is in glycated form with a structural formula:

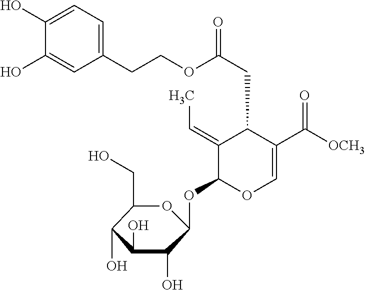

and oleuropein derivatives are made up of oleuropein aglycone, with a structural formula:

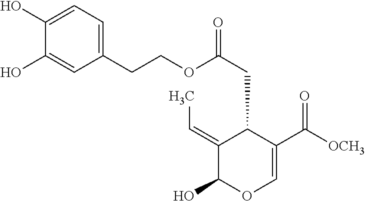 [SMILES: C/C=C1/[C@H](O)OC=C(C(=O)OC)[C@H]1CC(=O)OCCc1ccc(O)c(O)c1]

and its metabolic derivatives, such as tyrosol and hydroxytyrosol, ligstroside, elenolic acid, oleacine and oleocanthal, with the formula:

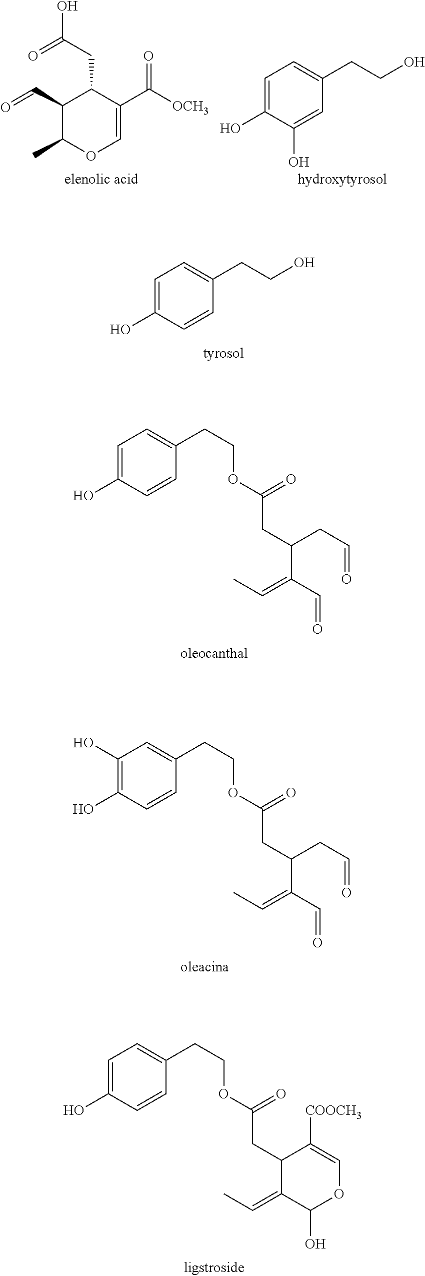 [SMILES: COC(=O)C1=CO[C@@H](C)[C@@H](C=O)[C@@H]1CC(=O)O ; OCCc1ccc(O)c(O)c1 ; OCCc1ccc(O)cc1 ; C/C=C(/C=O)C(CC=O)CC(=O)OCCc1ccc(O)cc1 ; C/C=C(/C=O)C(CC=O)CC(=O)OCCc1ccc(O)c(O)c1 ; C/C=C1/C(O)OC=C(C(=O)OC)C1CC(=O)OCCc1ccc(O)cc1]

In particular, glutathione is contained in appropriate quantities to ensure a daily intake of 30 to 1000 mg of glutathione and oleuropein or a derivative of it are contained in appropriate quantities to ensure a daily intake of between 50 and 1000 mg.

Advantageously, the pharmaceutical composition contains at least one or more pharmaceutically acceptable ingredients, having nootropic or neuroprotective properties, preferably selected from the following: curcumin, bacosides from Bacopa monnieri, vitamin D_{3,} vitamin B_{6,} astaxanthin, lutein, tocopherols, tocotrienols, vitamin B₁₂, choline, betaine.

The pharmaceutical composition is specifically aimed at the treatment or prevention of Alzheimer's disease, Parkinson's disease and other neurodegenerative diseases related to ageing and characterised by the progressive deterioration of neurological performance. The pharmaceutical composition can be made in the form of a drug, a food supplement, a medical device or a topical formulation to be administered orally, enterally, parenterally, transmucosally or transcutaneously.

Below are some detailed examples of the invention and of experiments carried out

### Example 1

### Activity on Cultured Cells

N2a (ECACC) mouse neuroblastoma cells, plated in the presence of MEM integrated with non-essential amino acids, 10% FCS, antibiotics and glutamine, were exposed for 48 h to micromolar levels of oleuropein aglycone and/or glutathione thioesters (GST), in the presence and in the absence of prefibrillar aggregates of Aβ1-42 peptides (Lambert M, et al. J. Neurochem. 2001, 79:595-605). In this experiment, S-linoleyl-glutathione, S-linolenoyl-glutathione, S-oleyl-glutathione and S-docohexaenoyl-glutathione, were used as thioesters of glutathione, with equivalent results. In the control plates (CTRL) the cells were incubated under the same conditions in the presence of the culture medium alone. The following were then evaluated:
the state of cell viability through the MTT reduction test (Pensalfini A et al., Free

Rad Biol Med 2008, 44: 1624-1636).
the level of mTOR substrate phosphorylation of kinase p70 S6 (mTOR P) by western blot analysis and subsequent densitometry (Grossi C, et al., PLoS ONE 2013, 8: e71702)
the level of intracellular antioxidant capacity (ACi) through hydrosolublescavenger doses in the cellular lysates in chemiluminescence, using
the Pholasin photoprotein (ABEL Antioxidant Test Kit, Knight Scientific Ltd., UK) as reported in Cecchi C., et al. Neurobiol. Aging 28: 863-876, 2007.

Table 1 shows the results obtained from these experiments. ACi values were correct for the % of viable cells. AP=toxic prefibrillar aggregates.

TABLE 1

| | | Aβ42 | OLE | OLE 2 □M + | GST | GST 3 □M + | OLE 2 □M + | OLE 2 □M + GST |
|---|---|---|---|---|---|---|---|---|
| | | AP 3 | 2 | Aβ42 AP | 3 | Aβ42 AP | GST 3 | 3 □M + Aβ42 |
| | CTRL | μM | □M | 3 μM | □M | 3 uM | □M | AP 3 μM |
| Cell viability | NA | 40* | 98 | 65* | 97 | 71* | 104 | 96 |
| (MTT test), % | | | | | | | | |
| respect to controls | | | | | | | | |
| mTOR P, % respect | NA | 135** | 12* | 69 | 118 | 102 | 16* | 45* |
| to controls | | | | | | | | |
| ACi, mmol/L GSH | 2.60 | 1.22* | 2.68 | 1.55* | 3.02** | 1.73* | 3.05** | 2.47 |
| Equivalent | | | | | | | | |

*p < 0.001
**p < 0.05

Similar results have been obtained with experiments conducted with human neuroblastoma SY5Y cells treated under the same conditions and show how of autophagy stimulation, in these experimental conditions, is mediated by the inhibition of mTOR. No significant differences were observed between the results obtained using glycated oleuropein, oleuropein aglycone or its metabolic derivative hydroxytyrosol. The enhancing effect with respect to the reduction in cytotoxicity and the increase in intracellular antioxidant capacity resulting from the synergistic action of the thioesters of glutathione with oleuropein is also remarkable.

### Example 2

### Measurement of Cognitive-Behavioural Performance in Animal Models

As an animal model of AD, CRND8 transgenic mice encoding a double mutant of APP695 [28] and wild-type (WT) control mice (Chishti M A, et al., J Biol Chem 2001, 276: 21562-21570) were used, in accordance with Legislative Decree 116/92 and EU 2010/63 directive.

Mice groups (equally divided by gender) of 3 months WT (n=12) and TG (n=12) were fed with different diets for rodents (Lab Diet Roditor standard diet, RSD) for eight weeks:
A: 5% lipid standard rodent diet (10 g/day per individual).
B: 5% lipid standard rodent diet (10 g/day per individual) containing OLE (30 mg/kg of diet).
C: 5% lipid standard rodent diet (10 g/day per individual) containing SAG (30 mg/kg of diet).
D: 5% lipid standard rodent diet (10 g/day per individual) containing LCFA-SG (50 mg/kg of diet).
E: 5% lipid standard rodent diet (10 g/day per individual) containing OLE (30 mg/kg of diet) and SAG (50 mg/kg of diet).
F: 5% lipid standard rodent diet (10 g/day per individual) containing OLE (30 mg/kg of diet) and LCFA-SG (S-linolenoyl glutathione, 50 mg/kg of diet).

The RDS diet provides 30% energy from proteins, 14% from lipids, 56% from carbohydrates, and contains 5.3% of fibre.

At the end of the diet treatment the animals underwent tests for cognitive-behavioural performance, using the step-down avoidance test (Bellucci A, et al. Neurobiol. Dis. 2006, 23: 260-272) and the object recognition test (ORT, Greco SJ, et al., J Alzheimer's Dis 2010, 19: 1155-1167).

Table 2 shows the results obtained with the “Step-Down” test and the ORT test.

TABLE 2

| | | Test “Step-Down” | | | |
|---|---|---|---|---|---|
| | | | Significance* (with | Test ORT | |
| | | | respect to the | | Significance* (with |
| | | Latency | homogeneous | | respect to the |
| | | (Retention test) | genotype fed with | Discrimination | homogeneous genotype |
| Treatment | Genotype | sec. | diet A) | Index | fed with diet A) |
| A | WT | 160 | NA | 0.65 | |
| | TG | 15 | P < 0.001 | 0.32 | P < 0.001 |
| | | | (respect to WT) | | (respect to WT) |
| B | WT | 167 | P > 0.05 | 0.69 | P > 0.05 |
| | TG | 95 | P < 0.001 | 0.45 | P < 0.05 |
| C | WT | 165 | P > 0.05 | 0.67 | P > 0.05 |
| | TG | 65 | P < 0.001 | 0.40 | P < 0.05 |
| D | WT | 165 | P > 0.05 | 0.69 | P > 0.05 |
| | TG | 73 | P < 0.001 | 0.43 | P < 0.05 |
| E | WT | 172 | P > 0.05 | 0.69 | P > 0.05 |
| | TG | 145 | P < 0.001 | 0.63 | P < 0.001 |
| F | WT | 177 | P < 0.05 | 0.71 | P > 0.05 |
| | TG | 155 | P < 0.001 | 0.65 | P < 0.001 |

*The differences between each of the E-TG and F-TG groups and each of the B-TG, C-TG and D-TG groups were also highly significant.

### Example 3

### Evaluation of Cognitive Performance in Humans

Seven adult subjects, three males and four females, of which five were Caucasian and two Asian, between the ages of 63 and 78, classified as MCI subjects according to international guidelines (Neurologist. 2012;18(6):356-363.) took 1 daily dose of a preparation (A), within a varied, balanced and complete diet for 180 days. The preparation (A) contained:

| | |
|---|---|
| S-acetyl glutathione | 120 mg |
| N-acetyl cysteine | 100 mg |
| oleuropein | 100 mg |
| Vit D | 10 μg |
| Vit B12 | 2 μg |
| Vit E (a-tocopherol) | 20 mg |
| Bacosides (from B. monnieri) | 100 mg |
| Piperine | 2 mg |

Another homogeneous group of individuals, in terms of number, age, gender, ethnicity and diagnosis took 1 daily dose of a preparation (B) during the same period, within a varied, balanced and complete diet. The preparation (B) contained:

| | |
|---|---|
| N-acetyl cysteine | 100 mg |
| Vit D | 10 μg |
| Vit B12 | 2 μg |
| Vit. E (a-tocopherol) | 20 mg |
| Bacosides (from B. monnieri) | 100 mg |
| Piperine | 2 mg |

At 0, 3 and 6 months from the start of the nutritional treatment, the GPCog test was administered to individuals of both groups (Pirani A et al., Int. Psychogeriatrics 2010, 22: 82-90).

Table 3 shows the results of the test, expressed as average scores.

TABLE 3

| | t = 0 d | | | | t = 90 d | | | |
|---|---|---|---|---|---|---|---|---|
| | Section 1 | Sig- | Section 2 | Sig- | Section 1 | Sig- | Section 2 | Sig- |
| | (patient | nifi- | (informant | nifi- | (patient | nifi- | (informant | nifi- |
| | examination) | cance | interview | cance | examination) | cance | interview) | cance |
| Test Group | 4.1 | P > | 2.8 | P > | 6.9 | P < 0.05 | 4.7 | P < 0.001 |
| (preparation | | 0.05 | | 0.05 | | (vs. B) | | (vs. B) |
| A) | | (vs. B) | | (vs. B) | | P < 0.001 | | P < 0.001 |
| | | | | | | (vs. 0 d) | | (vs. 0 d) |
| Control | 4.5 | NA | 2.3 | NA | 4.6 | NA | 2.5 | NA |
| Group | | | | | | | | |
| (preparation | | | | | | | | |
| B) | | | | | | | | |
| | | | | | t = 180 d | | | |
| | | | | | Section 1 | Sig- | Section 2 | Sig- |
| | | | | | (patient | nifi- | (informant | nifi- |
| | | | | | examination) | cance | interview) | cance |
| | | | | | 7.2 | P < 0.001 | 4.6 | P < 0.001 |
| | | | | | | (vs. B) | | (vs. B) |
| | | | | | | P < 0.001 | | P < 0.001 |
| | | | | | | (vs. 0 d) | | (vs. 0 d) |
| | | | | | 4.1 | NA | 1.9 | NA |

At times t=0 and t=180 d a skin prick capillary blood test was performed on the individuals of the two groups, in order to evaluate the total antioxidant capacity (Ciuti R et al., ELAS Proceedings, Bologna 2013; J. Biomedical Science and Engineering, 2017, 10, 60-76). Table 4 shows the results obtained, expressed in terms of mmol/L of equivalent glutathione.

TABLE 4

| | t = 0 | Significance | t = 180 d | Significance |
|---|---|---|---|---|
| Test Group | 1.4 | p > 0.05 | 2.3 | P < 0.001 |
| (preparation A) | | (vs B) | | |
| Control Group | 1.2 | NA | 1.3 | NA |
| (preparation B) | | | | |

The evidence described was obtained from the use of a preparation based on glutathione thioester and oleuropein, also potentially in association with other bioactive principles with antioxidant activity and with neurotrophic, nootropic and neuro-protective potential, such as extracts of Bacopa monnieri, astaxanthin, phosphatidylserine, vitamin E, etc. The evidence constitutes, according to the present invention, the rationale for the use of pharmaceutical preparations and nutritional supplements that are useful in counteracting or slowing cognitive decline, acting synergistically on the neuropathophysiological processes that form the basis of this.

1. Pharmaceutical composition for prevention and treatment of human cognitive deficits that comprises oleuropein or oleuropein derivatives and glutathione/carboxylic acid conjugate, having a molecular formula: 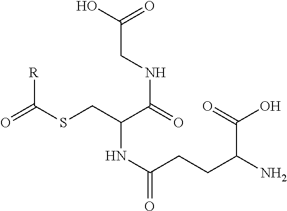
where R=residue of aliphatic carboxylic acid,
wherein the carboxylic acid component of the glutathione/carboxylic acid conjugate is represented by any of the saturated or unsaturated aliphatic carboxylic acid, containing from 2 to 22 carbon atoms.

2. Pharmaceutical composition according to claim 1, wherein the carboxylic acid component of the glutathione/carboxylic acid conjugate is represented by ethanoic acid.

3. Pharmaceutical composition according to claim 1, wherein the carboxylic acid component of the glutathione/carboxylic acid conjugate is represented by a poly-unsaturated aliphatic carboxylic acid containing 18 carbon atoms, comprising an omega-3, omega-6 or omega-9 series acid.

4. Pharmaceutical composition according to claim 1, wherein the oleuropein is in glycated form having the structural formula: 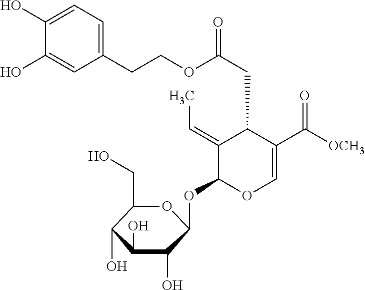
and the oleuropein derivatives consist of oleuropein aglycone, with the molecular formula: 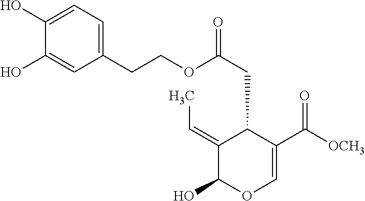 [SMILES: C/C=C1/[C@H](O)OC=C(C(=O)OC)[C@H]1CC(=O)OCCc1ccc(O)c(O)c1]
and oleuropein aglycone metabolic derivatives, comprising tyrosol and hydroxytyrosol, ligstroside, elenolic acid, oleacin and oleocanthal, having molecular formula:

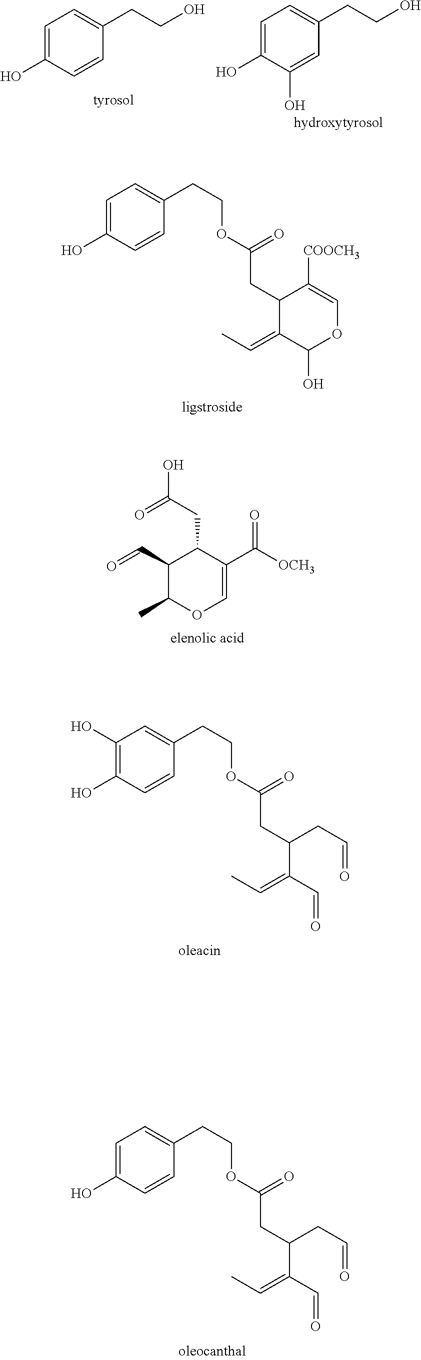 [SMILES: OCCc1ccc(O)cc1 ; OCCc1ccc(O)c(O)c1 ; C/C=C1/C(O)OC=C(C(=O)OC)C1CC(=O)OCCc1ccc(O)cc1 ; COC(=O)C1=CO[C@@H](C)[C@@H](C=O)[C@@H]1CC(=O)O ; C/C=C(/C=O)C(CC=O)CC(=O)OCCc1ccc(O)c(O)c1 ; C/C=C(/C=O)C(CC=O)CC(=O)OCCc1ccc(O)cc1]

5. Pharmaceutical composition according to claim 1, wherein the glutathione is contained in an amount appropriate to ensure a daily intake of 30 to 1000 mg of glutathione, and oleuropein or a derivative thereof are contained in appropriate quantities to ensure a daily intake of between 50 to 1000 mg.

6. Pharmaceutical composition according to claim 1 containing at least one or more pharmaceutically acceptable ingredients eliciting nootropic or neuro-protective effects, selected from the following: curcumin, bacosides from Bacopa monnieri, vitamin D3, vitamin B6, astaxanthin, lutein, tocopherols, tocotrienols, vitamin B12, choline, betaine.

7. Pharmaceutical composition according to claim 1 for use in the treatment or prevention of Alzheimer's disease, Parkinson's disease and of other related neurodegenerative diseases and characterized by progressive detriment of neurological performance.

8. Pharmaceutical composition according to claim 1 in the form of medication, food supplement, medical device or topical formulation to be administered via oral, enteral, parenteral, trans-mucosal or transcutaneous route.
